(19) United States
(12) Patent Application Publication
(10) Pub. No.: US 2023/0321304 A1
(43) Pub. Date: Oct. 12, 2023

(54) REFILLABLE VOLATILE COMPOSITION DISPENSER WITH RELEASABLE INTERLOCK

(71) Applicant: The Procter & Gamble Company, Cincinnati, OH (US)

(72) Inventors: Rahul VYAS, Singapore (SG); Desmond NG, Singapore (SG); Mei San Gigi CHO, Singapore (SG); William Michael CANNON, West Harrison, IN (US)

(21) Appl. No.: 18/128,274

(22) Filed: Mar. 30, 2023

Related U.S. Application Data
(60) Provisional application No. 63/329,485, filed on Apr. 11, 2022.

(51) Int. Cl. A61L 9/12; A61L 9/04

(52) U.S. Cl. CPC A61L 9/12; A61L 9/048

(57) ABSTRACT

A flexible solid deodorizer for releasably interlocking to a volatile composition dispenser, the solid deodorizer has a first central evaporative surface, a second central evaporative surface, and a peripheral evaporative surface. The peripheral evaporative surface circumscribes at least a part of the first and second central evaporative surfaces to define a side of the deodorizer. The deodorizer is configured for releasably interlocking to the dispenser, wherein at least a portion of the deodorizer is configured to releasably interlock with at least a portion of the dispenser to form a solid surface substantially free of an opening.

### FIELD OF THE INVENTION

The present invention relates to a volatile composition dispenser for delivering a volatile composition, and particularly relates to refillable volatile composition dispenser comprising a solid deodorizer and a releasable interlock.

### BACKGROUND OF THE INVENTION

The use of various devices for diffusion of volatile compounds such as perfumes, sanitizing agents, insect repellants, air freshening compositions, deodorizers are well known. For example, consumers buy air fresheners to freshen their homes. Conventional air fresheners release an air freshening composition into the surroundings for a period of time until the freshening composition is depleted. Various types of refillable air fresheners are also well known in the state of the art. Such refillable air fresheners may utilize a liquid composition, a solid composition or a gel composition for delivering volatile compounds. Solid air fresheners such as gel air fresheners conventional gels have a gelling polymer which remains after water and perfume evaporates, and the remaining gelling polymer causes stickiness in the gel. Further, the remaining gel also stick to the container break easily and causes messy residue. As such, consumers need to clean the container before directly refilling the container with a new gel refill.

A refillable air freshening device is described in GB-A2374805 which comprises a primary container having a gel receiving surface having recesses for receiving a gel composition, and a refill container having a gel receiving surface and a refill container having a gel receiving surface profiled to correspond to the gel receiving surface of the primary container and also having recesses for receiving the gel. GB-A2374805 describes in page 7, lines 10 to 14 that once the gel composition in the original air freshener device has dissipated, and any residue removed from the container, the refill container can be positioned so that its rear surface abuts the gel receiving surface of the container.

WO2005027630A describes an air freshening device having a primary container and a refill container with a gel receiving surface having at least one recess for receiving a gel composition and is located and firmly held within the primary container. However, when the gel composition in such refill containers have dissipated over time, the refill containers will still be discarded. Accordingly, there remains a need for a refillable volatile composition dispenser for a solid deodorizer that is easy to refill, without the need of a refill container.

### SUMMARY OF THE INVENTION

The present invention relates to a flexible solid deodorizer for releasably interlocking to a volatile composition dispenser, the solid deodorizer comprising: a first central evaporative surface, a second central evaporative surface, and a peripheral evaporative surface, wherein the peripheral evaporative surface circumscribes at least a part of the first and second central evaporative surfaces to define a side of the deodorizer; wherein the deodorizer is configured for releasably interlocking to the dispenser, wherein at least a portion of the deodorizer is configured to releasably interlock with at least a portion of the dispenser to form a solid surface substantially free of an opening.

### BRIEF DESCRIPTION OF DRAWINGS

FIG. 1 is a front perspective view of a solid deodorizer according to the present invention;

FIG. 2 is a side view of the solid deodorizer of FIG. 1;

FIG. 3 is an exploded assembly view of a volatile composition dispenser according to the present invention with the solid deodorizer of FIG. 1;

FIG. 4 is a front schematic view of the volatile composition dispenser of FIG. 3;

FIG. 5 is a front perspective view of components of an alternative design of a volatile composition dispenser according to the present invention;

FIG. 6 is a front perspective view of an assembled volatile composition dispenser of FIG. 5;

FIG. 7 is a perspective view of components of a volatile composition dispenser according to the present invention.

FIG. 8 is a front perspective view of the volatile composition dispenser of FIG. 7 in a first configuration in which the solid deodorizer has an initial size; and

FIG. 9 is a front view of the volatile composition dispenser of FIG. 7 in a second configuration in which the solid deodorizer has a reduced size as a result of shrinkage.

### DETAILED DESCRIPTION OF THE INVENTION

It has been found that refillable consumer products play an important role in enabling consumers/users to have a choice in deciding to make a new purchase or to refill a packaging container of a depleted product. In particular, it is desirous for users of volatile dispensing products for delivering a benefit in an interior space to refill a container of a volatile dispensing product when the volatile composition is exhausted.

The present invention relates to a flexible solid deodorizer for releasably interlocking to a volatile composition dispenser, the solid deodorizer comprising: a first central evaporative surface, a second central evaporative surface, and a peripheral evaporative surface, wherein the peripheral evaporative surface circumscribes at least a part of the first and second central evaporative surfaces to define a side of the deodorizer; wherein the deodorizer is configured for releasably interlocking to the dispenser, wherein at least a portion of the deodorizer is configured to interlock with at least a portion of the dispenser to form a solid surface substantially free of an opening.

A technical effect is that the solid deodorizer may be easily detached from the dispenser by exerting a pressure on the first central evaporative surface or the second central evaporative surface thereby releasing the at least a portion of the deodorizer from the interlock with the at least a portion of the dispenser. The at least a portion of the deodorizer may be configured for releasably locking to the at least a portion of the dispenser by one of: interference fit, pressure fit, friction fit, and combinations thereof.

In the following description, the solid deodorizer described is a consumer product, such as an air freshener, for evaporating a volatile composition in spaces to deliver a variety of benefits such as freshening, malodor removal or scenting of air in spaces such as rooms in household and commercial establishments, or enclosed spaces such as a vehicle passenger compartment space. However, it is contemplated that the solid deodorizer may be configured for use in a variety of applications to deliver a volatile composition to the atmosphere and the solid deodorizer may include but is not limited to consumer products, such as, for example air freshening products.

Prior to describing the present invention in detail, the following terms are defined for clarity. Terms not defined should be given their ordinary meaning as understood by a skilled person in the relevant art.

“Horizontal orientation” as used herein, refers to a position of a volatile composition dispenser according to the present invention wherein a central evaporative surface is facing the environment in an upward or downward position.

“Solid deodorizer” as used herein, refers to a chemically cross-linked gel composition that is molded in the form of a three-dimensional object having a width, length and thickness along an x-axis, y-axis, and z-axis, respectively. The solid deodorizer is self-supporting and comprises at least two evaporative surfaces.

“Visual indicator” as used herein, refers to any element that indicates a stage in the product life cycle of a volatile composition dispenser, or a state of use of the volatile composition dispenser.

“Visually perceptible” as used herein, refers to the ability to see the visual indicator and notice information represented by the visual indicator.

“Vertical orientation” as used herein, refers to a position of a volatile composition dispenser according to the present invention wherein a central evaporative surface is facing the environment in a forward-facing position or in a rear facing position.

“Non-energized” as used herein, means that the volatile composition dispenser is passive and does not require to be powered by a source of external energy. In particular, the volatile composition dispenser does not need to be powered by a source of heat, gas, or electrical current, and the volatile composition is not delivered by aerosol means. Further, as used in this specification and the appended claims, the singular forms “a”, “an”, and “the” include plural references unless the content clearly dictates otherwise.

“Top notes” as used herein, refer to perfume raw materials having a high volatility.

“Bottom notes” as used herein, refer to perfume raw materials which are less volatile relative to the top notes.

“Vapor” as used herein, refers to a gaseous form of an organic or inorganic substance which coexists as a solid or liquid at ambient conditions including but not limited to temperature, humidity, and air pressure.

“Vapor impermeable substrate” as used herein, refers to a material configured to resist diffusion of vapor from the dispenser prior to its intended use.

“Vapor release rate” as used herein, refers to a measure of the passage of vapor through a substrate.

“Volatile composition” as used herein, refers to a material that is vaporizable at room temperature and atmospheric pressure without the need of an additional energy source. The volatile composition may be configured for various uses, including but not limited to, air freshening, deodorization, odor elimination, malodor counteraction, pest control, insect control, insect repelling, medicines/medicaments, disinfectants, sanitization, mood enhancement, aromatherapy aid, scented compositions, non-scented compositions, or any other use which requires a volatile composition that acts to condition, modify, or otherwise change the atmosphere or the environment. Further, it is not necessary for all of the component materials of the volatile composition to be volatile. Any suitable volatile composition in any amount or form, including a liquid, solid, gel or emulsion, may be used. Materials suitable for use herein may include non-volatile compounds, such as carrier materials (e.g., water, solvents, etc.). It should also be understood that when the volatile composition is described herein as being “delivered”, “emitted”, or “released”, this refers to the volatization of the volatile component thereof and does not require that the non-volatile components thereof be emitted.

For the purposes of illustrating the present invention in detail, the invention is described below as a solid deodorizer comprising a volatile composition and a non-energized volatile composition dispenser. However, the volatile composition dispenser may be configured for use with an energized device such as, for example, an electrical heating device or a fan. The solid deodorizer described hereinafter is a gel composition comprising a perfume as an example of a volatile composition. The gel composition is polyester polyol cross-linked using a cross-linking agent selected from the group consisting of: isocyanates, isothiocynates and mixtures thereof. However, it will be appreciated that the solid deodorizer may be formed from any gel composition that can be molded into three-dimensional objects and that when set, is self-supporting and has at least a peripheral evaporative surface and a central evaporative surface.

The solid deodorizer may be configured in a variety of shapes and sizes to facilitate customization for use as volatile composition dispensers such as air fresheners in vehicles, residential interior spaces, commercial interior spaces, a household furniture interior space such as cupboards or lockers, a household appliance interior space. Preferably the household appliance may be selected from the group consisting of: refrigerators, air conditioners, washing machine, automatic dishwashing machine. The interior space may be a portable consumer product interior environment, preferably the portable consumer product may be bags, luggage or the like.

The solid deodorizer of the present invention can be implemented using a volatile composition dispenser, such as an apparatus for delivering a volatile composition into an interior space. It is contemplated that the apparatus may be configured for use in a variety of applications to deliver volatile materials to the atmosphere and/or a surface as long as the volatile material is evaporated from the apparatus. For the purposes of this disclosure, but without intending to limit the scope of the invention, the apparatus is described as a non-energized apparatus.

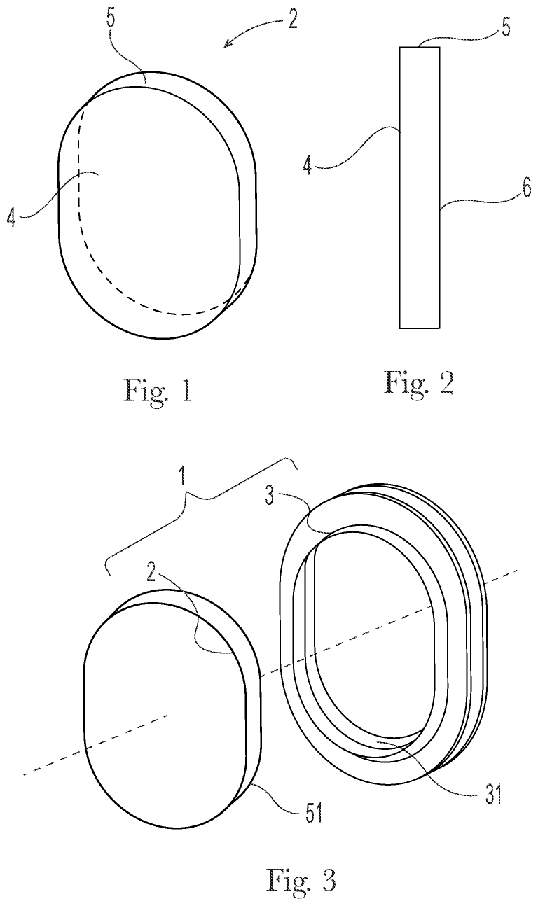

FIG. 1 is a front view of a solid deodorizer 2 comprising a volatile composition according to the present invention. FIG. 2 is a side view of the solid deodorizer of FIG. 1. The solid deodorizer 2 has a length L, width W and thickness D defining a three-dimensional self-supporting structure of the solid deodorizer 2. The solid deodorizer 2 may be characterized by a thickness D of from 0.5 mm to 15 mm, from 1 mm to 10 mm, from 2 mm to 9 mm, from 3 mm to 8 mm, or different combinations of the upper and lower values described above or combinations of any integer in the ranges listed above.

The solid deodorizer 2 is configured to contain a solid phase of the volatile composition and comprises a plurality of evaporative surfaces to allow the solid phase of the volatile composition to evaporate therefrom. The volatile composition may be selected from the group consisting of: a perfume, a deodorizing agent, a sanitizing agent, an insect repellant, a malodor reduction agent, and mixtures thereof The volatile composition may be present in a level of 3 wt % to 85 wt %, preferably from 15 wt % to 75 wt %, more preferably from 20 wt % to 60 wt %, or different combinations of the upper and lower percentages described above or combinations in the ranges listed above, by weight of the solid deodorizer.

Referring to FIG. 1, the solid deodorizer 2 comprises a first central evaporative surface 4, a second central evaporative surface 6, and a peripheral evaporative surface 5 surrounding the first and second central evaporative surfaces 4, 6. Referring to FIG. 2, the peripheral evaporative surface 5 circumscribes at least a part of the first and second central evaporative surfaces 4, 6 to define a side of the deodorizer 2. The deodorizer 2 is configured for releasably interlocking to a volatile composition dispenser, wherein at least a portion of the deodorizer is configured to interlock with at least a portion of the dispenser to form a solid surface substantially free of an opening.

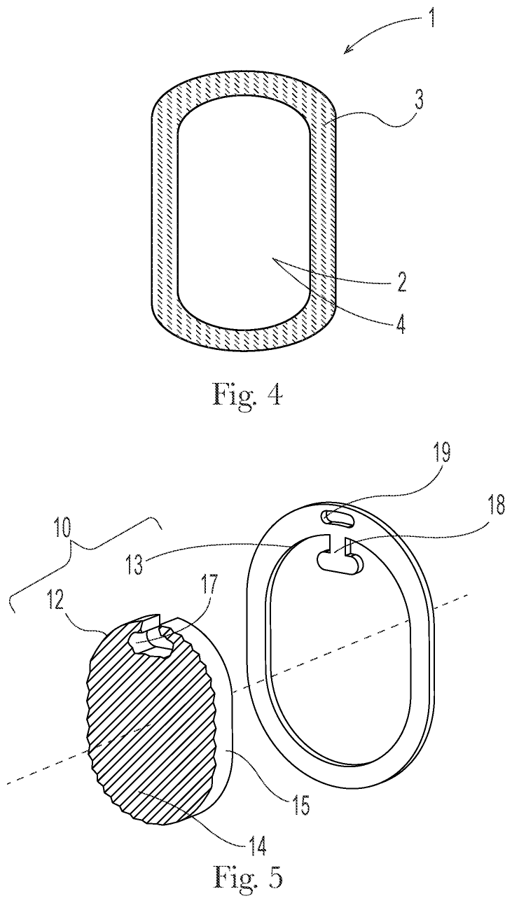

FIG. 3 is an exploded assembly view of a volatile composition dispenser 1 according to the present invention. The dispenser 1 comprises a housing 3 and the solid deodorizer 2 of FIG. 1. Specifically, the peripheral evaporative surface 5 of the solid deodorizer 1 defines an outer profile 51. To releasably interlock the deodorizer 2 to the dispenser 1, a shape of an outer profile 51 of the solid deodorizer 2 may correspond to an inner profile 31 of the housing 3. Each of the first and second central evaporative surface 4, 6 is characterized by a central evaporative surface area larger than a peripheral evaporative surface area of the peripheral evaporative surface 5. The deodorizer 2 may be releasably detached from the housing 3 by applying a finger pressure on the first central evaporative surface 4 or the second central evaporative surface 6. A technical effect is that there is minimum contact area of the deodorizer 2 in that only the peripheral surface 5 is in contact with the inner profile 31 of the housing 3 thereby reducing a size of surface areas that can have residue.

FIG. 4 is a front schematic view of the dispenser 1 of FIG. 3. As shown in FIG. 4, the size and shape of the outer profile 51 of the deodorizer 2 is configured to mate with the size and shape of the inner profile 31 of the housing 3 so as to form a continuous surface substantially free of a gap so that the deodorizer 2 can be secured within the housing 3. The solid deodorizer 2 can be made of any known material that is capable of providing a self-supporting structure and having a mixture of evaporable ingredients and non-evaporable ingredients, as long as the amounts of the evaporable ingredients and non-evaporable ingredients are configured to provide shrinkage of greater than 1% to less than 40%. Evaporable ingredients may include a volatile composition and water. Non-evaporable ingredients may include ingredients suitable for forming a solid deodorizer including but not limited to gelling materials, elastomers, polyurethane or the like.

Table 1 shows an example of a water-based gel composition according to the present invention.

TABLE 1
Example of a Water-based gel composition

| | | Target (%) |
|---|---|---|
| | | by weight |
| | | of the |
| Ingredient | Function | composition |
| Water | Solvent | 35 |
| Carrageenan and NaCl | Gelling material | 25 |
| Mixture | | |
| Gellan Gum | Gelling material | 22 |
| Calcium Chloride | Stabilizer | 15 |
| Perfume | Fragrance | 1 |
| Hydrogenated Castor | Emulsifier | 2 |
| Oil | | |

The solid deodorizer may be non-aqueous, preferably comprises less than 1% by weight of water, more preferably substantially free of water.

The solid deodorizer may comprise a material selected from the group consisting of: an ethyl cellulose polymer, a chemically cross-linked polyol or derivative thereof, and mixtures thereof. Table 2 shows an example of a non-aqueous gel composition comprising an ethyl cellulose polymer which is capable of having a shrinkage of 1% to 40%.

TABLE 2
Non-aqueous gel composition

| | Target Range |
|---|---|
| | (wt % by weight |
| | of the |
| Ingredient | composition) |
| Poly(propylene oxide), weight average | 20 to 75 |
| molecular weight of 300 to | |
| 1,000 Daltons | |
| Ethyl cellulose polymer | 0.5 to 30 |
| C₁₋₅ alkyl monoester of a C₈₋₂₂ | 20 to 75 |
| fatty acid | |
| Perfume | 4.5 to 50 |
| water | 0 to 1.5 |

The solid deodorizer may be a chemically cross-linked polyol, wherein the polyol or derivative thereof is selected from the group consisting of: polyol, polyester polyol, polyglycerol and mixtures thereof, preferably the polyol derivative is a polyester polyol, more preferably castor oil, even more preferably the polyester polyol is cross-linked using a cross-linking agent selected from the group consisting of: isocyanates, isothiocynates and mixtures thereof.

The solid deodorizer may also be molded with a moldable material such as any one of the non-aqueous gel compositions described hereinafter.

### Non-Aqueous Gel Composition:

The gel composition is formed using a cross-linking agent which forms covalent bonds which are stable mechanically and thermally, so once formed are difficult to break. In contrast, physical cross-links rely on changes in the microstructure to achieve stability, such as crystalline regions or regions of high entanglement.

While physical gels can also hold high levels of hydrophobic material such as perfume, the processing of such physical gels is more delicate, as they are more readily broken during manipulation. In addition, such physical gels typically exhibit larger reductions in volume as the hydrophobic material evaporates, in comparison to the cross-linked gels of the present invention, typically at the level of from 50% to 90% by length reduction as the hydrophobic material evaporates. In contrast, the cross-linked gels of the present invention exhibit less shrinkage as the hydrophobic material is released, typically of the order of from 1% to 40%, preferably from 3% to 30%, more preferably from 4% to 20%, at the end of a time period of 1 to 75 days, preferably from 1 to 60 days, more preferably from 1 to 45 days.

The gel composition can have an elastic modulus G′ of above 0.1 kPa, preferably above 1 kPa, even more preferably above 2 kPa, and below 100 kPa.

The gel can be a chemically cross-linked polyol or derivative thereof. Suitable polyols or derivatives thereof can be selected from the group consisting of: polyol, polyester polyol, polyglycerol and mixtures thereof. Polyols, polyester polyols and polyglycerols comprise multiple hydroxyl groups, and are suitable for forming gels having a compact network. In addition, the resultant gel has greater affinity for hydrophobic materials which are less strongly hydrophobic.

Suitable polyols or derivatives thereof can have a molecular weight of from 60 Da to 10000 Da, preferably from 150 Da to 3000 Da, even more preferably from 500 Da to 2000 Da, even more preferably 600 Da to 1300 Da. Longer polyols and derivatives thereof, result in greater flexibility of the gel.

Suitable polyols and derivatives thereof do not comprise terminal hydroxyl groups. Secondary alcohols are particularly suitable. Primary alcohols, having terminal hydroxyl groups, typically result in more linear chains and a more compact network. A combination of primary and secondary alcohols are preferred, since they result in a more desired correlation length.

An average correlation length of less than 8 nm as measured using Small Angle X-Ray Scattering (SAXS) is preferred. However, the gel compositions, described herein, can be formulated to have any desired correlation length.

A gel with more optimal pore size is achieved when secondary alcohols are used. Lightly branched polyols and derivatives thereof, such as poly(diethyleneglycol adipates) result in more flexible gels. Preferred polyols and derivatives thereof have at least 2 hydroxyl groups per molecule, more preferably at least 3 hydroxyl groups per molecule.

A polyol is a compound containing multiple hydroxyl groups. Diol polyols, having two hydroxyl-functional groups, result after cross-linking in linear polymers or more open networks having large pore size. In contrast, hydroxyl-functional monomers with functionality larger than two form more compact gels with smaller pore sizes. Suitable polyols include: ethylene glycol, propylene glycol, 1,4-butanediol, 1,6-hexanediol, pentaerythritol, 1,2,6-hexanetriol, 4,6-di-tert-butylbenzene-1,2,3-triol, propanetriol (glycerol), 1,2,5-hexanetriol, 1,2,4-cyclohexanetriol, 2,5-dimethylhexane-1,2,6-triol, 3-hydroxymethylpentane-1,2,5-triol, 1,3,6-hexanetriol, 1,1,5,5-pentanetretraol, 1,2,5,6-hexanetretraol, 1,2,3,4,5,6-hexanehexol (sorbitol) and mixtures thereof. Polyester polyols are hydroxyl-containing esters. Suitable polyester polyols can be selected from the group consisting of: aliphatic polyester polyols, aromatic polyester polyols, organic oil based polyester polyols, and mixtures thereof. Organic oil based polyester polyols are preferred. Preferred organic oils are vegetable oils since they typically comprise high levels of unsaturation (C═C bonds) and naturally comprise hydroxyl groups. Suitable polyester polyols include: hexanoic acid, 4-hydroxy-, 1,1′,1″-(1,2,3-propanetriyl) ester; pentanoic acid, 5-amino-4-hydroxy-, 1,1′,1″-(1,2,3-propanetriyl) ester; Polycaprolactone triol; castor oil, hydroxyl sunflower oil (HSO) and mixtures thereof.

Castor oil is particularly suitable. Castor oil (Ricinus oil) is a pale yellow and viscous liquid, derived from the bean of the castor plant (Ricinus communis). Castor oil is predominately made up of triglycerides of fatty acids that contain 87-90% of ricinoleic acid (cis-12-hydroxyoctadec-9-enoic acid) and can be achieved in high purity grades. Castor oil and its derivatives have been used as polyols for polyurethanes and adhesives. The castor oil can be partially hydrogenated. It has been found that castor oil provides the length of the branches and the position of the hydroxyl groups which is particularly suited for providing a chemically cross-linked gel having a pore size which results in slow release of the hydrophobic material, particularly where the hydrophobic material is a perfume. In addition, the chemically cross-linked gels derived from castor oil show less syneresis of the hydrophobic material from the gel.

Polyglycerols are hydroxy-containing ethers. Polyglycerols are typically obtained by the polymerization of alkylene oxides (such as epoxides). Suitable alkylene oxides include ethylene oxide, propylene oxide, butylene oxide, and mixtures thereof, using chain initiators such as ethylene glycol, propylene glycol, diethylene glycol, dipropylene glycol, 1,4-butanediol, neopentyl glycol, pentaerythritol, hexanetriol, sorbitol, glycerol, and mixtures thereof. Suitable polyglycerols can be selected from the group consisting of: α,α-diglycerol, α,β-diglycerol, hyperbranched polyglycerol, dendritic polyglycerol, and mixtures thereof. Hyperbranched polyglycerols are aliphatic polyethers with multiple hydroxyl end groups that are obtained from the nonsymmetric polyaddition of glycidol to glycerol resulting in a globular branch-on-branch structure which provides special internal flexibility. Dendritic polyglycerols are a hyperbranched polyglycerol with a well-defined symmetric and spherical three-dimensional structure around a core. Apart from improving gel elasticity, the dendritic structure with sterically shielded core together with the exceptionally high number of functional groups of hyperbranched polyglycerols produces flexible gels with relatively low pore size, which increase the longevity of final composition by reducing the diffusion rate not only as a consequence of physically entrapping the hydrophobic material, but also enhancing H-bonding and Van der Waals interactions. Such polyglycerols can be purchased from Nanopartica GmbH (Germany) and Sigma-Aldrich. Suitable polyglycerols include: polyethylene glycol, polypropylene glycol, poly(diethylene glycol), poly(dipropylene glycol), poly(1,4-butanediol), poly(neopentyl glycol), poly(1,6-hexanediol), and mixtures thereof. The polyglycerol preferably has from 2 to 50, preferably from 4 to 30 repeat units.

Any suitable cross-linking agent can be used, though cross-linking agents selected from the group consisting of: isocyanates, isothiocynates and mixtures thereof, are preferred. The cross-linking agent can be a linear, branched, or cyclic isocyanate, and mixtures thereof. Cyclic isocyanates and mixtures thereof are preferred. Suitable cyclic isocyanates include heterocyclic isocyanates such as 1,3,5-tris(5-isocyanatopentyl)-1,3,5-triazine-2,4,6(1H,3H,5H)-trione.

Suitable cross-linking agents can be selected from the group consisting of: 1,4-butane diisocyanate (BDI), 1,6 hexamethylene diisocyanate (HMDI), L-Lysine ethyl ester diisocyanate (LDI), 4,4′-Methylenebis(cyclohexyl isocyanate) (H12MDI), Glycolide-ethylene glycol-Glycolide isocyanate (Bezwada, LLC), 4,4′-Methylenebis(phenyl isocyanate) (MDI), 2,4′-Methylenebis(phenyl isocyanate) (MDI), 2,2′-Methylenebis(phenyl isocyanate) (MDI), Isophorone diisocyanate (IPDI), 2,4-toluene diisocyanate (2,4-TDI), 2,6-toluene diisocyanate (2,6-TDI), Poly (hexamethylene diisocyanate) (PDI), 1,3-bis(2-isocyanatopropan-2-yl)benzene, Poly (pentamethylene diisocyanate) and mixtures thereof, preferably 1,6 hexamethylene diisocyanate (HMDI), L-Lysine ethyl ester diisocyanate (LDI), Poly (pentamethylene diisocyanate), Poly (hexamethylene diisocyanate) (PDI), 1,3,5-tris(5-isocyanatopentyl)-1,3,5-triazine-2,4,6(1H,3H,5H)-trione, and mixtures thereof. Such cross-linking agents are available from Sigma-Aldrich and from Covestro under trade name of Desmodur® eco N 7300.

The cross-linking agent can have a viscosity below 2.500 mPa·s at 25° C. and an isocyanate equivalent weight of from 15% to 40%, preferably from 18% to 30%. Such cross-linking agents are more easily blended with the polyol. As a result, more uniform gels can be achieved.

The gel is preferably essentially free, or free of unreacted isocyanates and/or isothiocyanates.

The gel can further comprise a hydroxyl containing polymer, a hydroxyl containing oligomer or mixtures thereof. The hydroxyl containing polymer and/or oligomer can be used to alter the elasticity of the gel composition, and therefore the longevity of the perfume release. since a higher elastic modulus G′ slows the perfume release.

Suitable hydroxyl containing polymers can be selected from the group consisting of: poloxamers, gelatins, carrageenan, chitin, chitosan, and mixtures thereof.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Suitable poloxamers have a weight average molecular weight of from 1500 g/mol to 15000 g/mol and a poly(ethylene oxide) weight percentage of from 10% to 80%, preferably from 50% to 80%. Suitable poloxamers are commercially available under the tradename of Pluronic® from BASF.

Gelatins are typically translucent, colorless, and typically obtained from collagen from various animal body parts. They are commonly used as a gelling agent in food, pharmaceutical industry, vitamin capsules, photography, and cosmetic manufacturing. Suitable gelatines can have a bloom of from 90 to 300. Bloom is a test to measure the strength of a gel or gelatin and is measured according to the method outlined by Bloom in U.S. Pat. No. 1,540,979. The test determines the weight in grams needed by a plunger with a diameter of 0.5 inch (12.7 mm) to depress the surface of the gel 4 mm without breaking it, at a temperature of 25° C. The result is expressed in Bloom (grades). It is usually between 30 and 300 Bloom. To perform the Bloom test on gelatin, a 6.67% by weight gelatin solution is kept for 17-18 hours at 10° C. prior to being tested.

Carrageenan are sulfated polysaccharide for instance derived from red algae, commonly known as Irish moss. They are typically composed principally of alpha-D-galactopyranose-4-sulfate units and 3,6-anhydro-alpha-D-galactopyranose units. At least three forms are known, designated, respectively, as “iota”, “kappa” and “lambda” carrageenan which differ in the ratios of the two galactopyranose units and accordingly in their sulfate ester content.

Kappa-carrageenan is the principal component in aqueous extracts from Chondrus crispus and Gigartina stellata. It is lower in sulfate ester content than iota and lambda carrageenan.

Chitosan is typically obtained by deacetylation under alkaline conditions of chitin, which is the second most abundant biopolymer in nature, after cellulose. Chitin can be found as an important constituent of the exoskeleton in animals, especially in crustaceans, molluscs and insects, and it is also the principal polymer in the cell wall of certain fungi. Chitin and chitosan are linear polysaccharides composed of randomly distributed β-(1-4)-linked D-glucosamine (deacetylated unit) and N-acetyl-D-glucosamine (acetylated unit). Chitosan has two types of reactive groups that can be grafted: the free amine groups on the deacetylated units and the hydroxyl groups on the C3 and C6 carbons on acetylated or deacetylated units.

The chitosan of the present invention may have a molecular weight from 10,000 g/mol to 4,000,000 g/mol, preferably from 70,000 g/mol to 1,600,000 g/mol. Suitable chitosan may have a degree of de-acetylation of at least 50%, preferably at least 60%, more preferably at least 70%, even more preferably at least 75%.

The gel composition can be transparent or even translucent. The gel composition can have any suitable shape, such as star, circular or pyramidal. The gel composition may be colored by adding dye. The gel compositions of the present invention can be moulded or even 3D printed to the desired shape.

The gel composition can be any suitable shape or size, since both define the evaporative surface area of the gel composition. It is known that the shape and size of the gel composition can affect the release and longevity of the hydrophobic material. For instance, thin sheets result in faster release and lower longevity than spheres of the same mass of the gel composition. Suitable gel compositions can have an evaporative surface area of less than 150 cm², preferably from 3.0 to 100 cm², more preferably from 6.0 to 60 cm².

The evaporative surface area can be measured by creating a 3D model of the gel composition using CAD software, and using the CAD software to calculate the surface area. Any suitable CAD software can be used, such as AutoCAD® 2013.

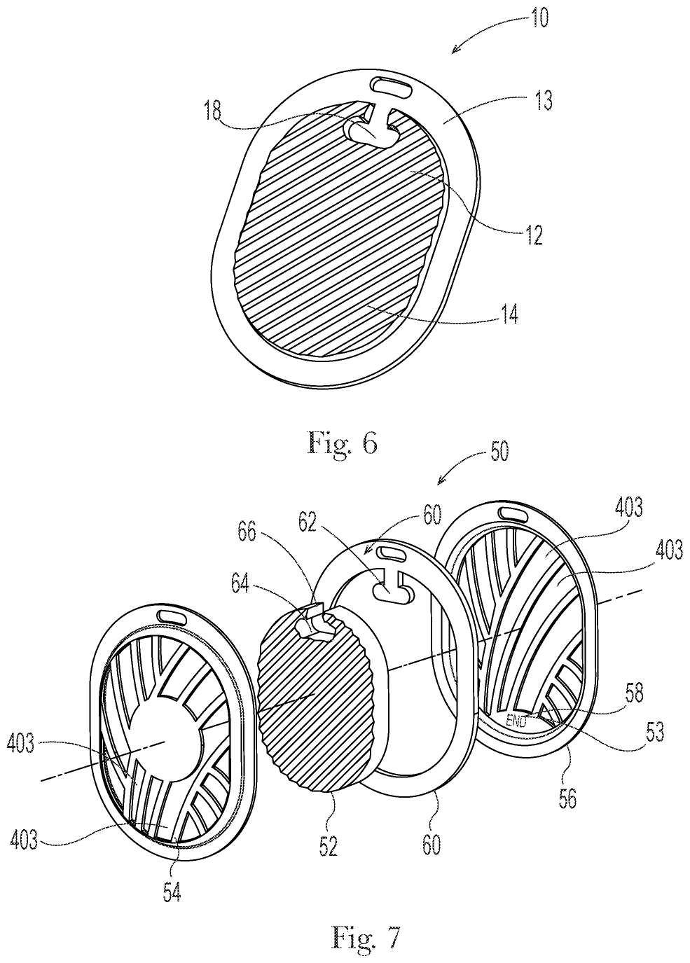

The total surface area of the first and second central evaporative surfaces 4 and 6 may be from 100 mm² to 10000 mm², from 1000 mm² to 8000 mm², from 1500 mm² to 6000 mm², from 2000 mm² to 4000 mm², or different combinations of the upper and lower values described above or combinations of any integer in the ranges listed above. An entire area of the first central evaporative surface 4 and/or the second central evaporative surface 6 may be non-textured such as shown in FIG. 1 and FIG. 2. Alternatively, a surface finish may be applied to the first central evaporative surface 4 such as shown in FIG. 5.

The surface finish may be a texture such as a matt finish, a 3-dimensional surface texture, or a combination of matt finish and 3-dimensional surface texture.

FIG. 5 is a front perspective view of an alternative embodiment of a volatile composition dispenser 10 with an article holder 13 and a solid deodorizer 12 according to the present invention. FIG. 6 is an assembled view of the dispenser 10. The solid deodorizer 12 comprises a first central evaporative surface 14, a second central evaporative surface (not shown), a peripheral evaporative surface 15 circumscribing the first and second central evaporative surfaces, and an anchor receiving component 17. The anchor receiving component 17 may be an aperture extending between the first and second central evaporative surfaces 14, 16 defining a through hole. The article holder 13 comprises an anchor 18 for releasably attaching to the anchor receiving component 17. The article holder 13 may further comprise a hanging tab 19 such that upon attachment to a mounting support, the article holder 13 supports the solid deodorizer 12 in a hanging configuration above a surface in an interior space, wherein the solid deodorizer 12 is releasably interlocked to the anchor 18 through the anchor receiving component 17.

FIG. 7 is a perspective view of components in a volatile composition dispenser 50 according to the present invention. The dispenser 50 comprises a solid deodorizer 52, an article holder 60 for the solid deodorizer 52 and a housing for housing the solid deodorizer 52 and the article holder 60. The housing 51 may comprise a front cover 54 and a rear frame 56, which upon assembly, the front cover 54 and the rear frame 56 define a housing interior for receiving the solid deodorizer 52. The article holder 60 may be disposed between the front cover 54 and rear cover 56 wherein the article holder 60 is configured for holding the solid deodorizer 52 in a predetermined position within the housing. Each of the front cover 54 and the rear frame 56 is provided with a plurality of openings 403 (hereinafter “opening”) for vaporizing of volatile composition in the solid deodorizer 52 into the interior space. The article holder 60 comprises an anchor 62 for releasably attaching the solid deodorizer 52 to the article holder 60, and a center opening 64 configured for circumscribing the solid deodorizer 52. The solid deodorizer 52 may comprise an aperture 64 sized and shaped to mate with the anchor 62. Specifically, the aperture 64 extends through an end part 66 of the solid deodorizer 52. Specifically, the housing 51 further comprises an inner surface 53, wherein a visual indicator 58 is disposed on the inner surface 53.

The housing, front cover, rear cover, and the article holder may be made of plastic, paper, or any material chemically compatible with the solid deodorizer.

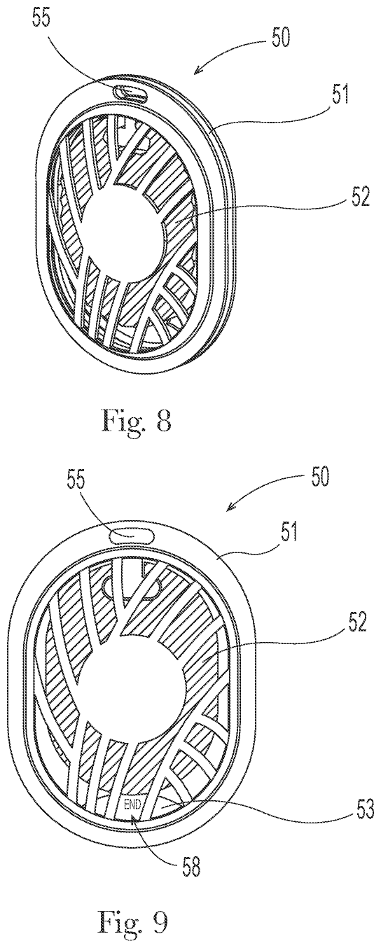

FIG. 8 is a front perspective view of the volatile composition dispenser 50. The solid deodorizer 52 has an initial size and may be configured with a texture to conceal the visual indicator 58 in a first configuration in which the visual indicator 58 is not visually perceptible according to the present invention.

FIG. 9 is a front view of the volatile composition dispenser 50 of FIG. 8 in a second configuration in which the visual indicator 58 is visually perceptible. Specifically, the solid deodorizer may be capable of shrinkage relative to the peripheral evaporative surface from an initial size to a reduced size smaller than the initial size upon exposure to the environment in an interior space.

A technical effect of shrinkage of the solid deodorizer 52 is that it provides a gap within the dispenser 50 for ease of removal from the article holder 60 after removing the front cover 54 and the rear cover 56.

The present invention also relates to a method of refilling a volatile composition dispenser with a solid deodorizer, the method comprising:
providing a volatile composition dispenser described hereinbefore;
applying a pressure on one of the first central evaporative surface and the second central
evaporative surface of the solid deodorizer to remove the solid deodorizer from the dispenser; and
releasably interlocking a second solid deodorizer to the dispenser.

A technical effect of the above method is that it is easy and intuitive for consumers to refill the volatile composition dispenser. The volatile composition dispenser may be an air freshener product, and the solid deodorizer may be an air freshening gel composition.

The following examples are intended to more fully illustrate the present invention and are not to be construed as limitations of the present invention since many variations thereof are possible without departing from the scope of the present invention. All parts, percentages and ratios used herein are expressed as percent weight unless otherwise specified.

### EXAMPLES

Data is provided demonstrating the volatile composition dispenser of the present invention having a solid deodorizer capable of shrinkage for release from the interlocking connection between the solid deodorizer and the dispenser for refillability. Solid deodorizers according to the present invention are prepared based on the composition details described in Table 3 below and are evaluated for the respective shrinkage (results shown in Table 2).

TABLE 3

| | | Inventive Sample (% by weight | | | | | |
|---|---|---|---|---|---|---|---|
| | | of the solid deodorizer) | | | | | |
| Component | Material | 1 | 2 | 3 | 4 | 5 | 6 |
| Polyester | Castor oil | 29 | 29 | 29 | 29 | 29 | 29 |
| Polyol | | | | | | | |
| Cross-linking | Isocyanate | 21 | 21 | 21 | 21 | 21 | 21 |
| Agent | | | | | | | |
| Volatile | Mixture of | 50 | 50 | 50 | 50 | 50 | 30 |
| Composition | perfume raw | | | | | | |
| | materials | | | | | | |
| Total | | 100 | 100 | 100 | 100 | 100 | 100 |

Inventive Samples of solid deodorizers made with the composition details described in Table 3 according to the process described below.

### Process of Making:

The polyol or derivative thereof, is mixed with the volatile composition, and optionally a hydroxyl containing polymer is added. Then, the cross-linking agent is added at a temperature from 5° C. to 35° C., preferably 15° C. to 30° C. and further mixed in order to provide a homogeneous mixture. The mixture is poured into a mould of the desired shape, and cured, preferably at a curing temperature from 20° C. to 30° C. Such temperatures limit the evaporation of volatile components of the hydrophobic material. Alternatively, the mixture can be kept at 5° C. or less, in order to avoid curing. Curing will then start only once the temperature is raised to the curing temperature.

The polyol or derivative thereof can be mixed with the cross-linking agent, and optionally a hydroxyl containing polymer, preferably at a temperature from 20° C. to 85° C., more preferably from 30° C. to 75° C., for 10 min to 10 hours, preferably from 15 min to 2 hours. The mixture is cooled down and the hydrophobic material is added preferably at a temperature from 10° C. to 40° C., more preferably 15° C. to 30° C. and can be further mixed, for instance, from 15 to 120 min. The mixture is poured into the desired mould and cured, preferably at a temperature from 20° C. to 30° C.

Alternatively, all of the components of the gel composition can be blended at a low temperature, such as 5° C. or less, before the temperature is increased to the curing temperature.

### Results

Shrinkage results of the Inventive Samples 1 to 6 of Table 3 are shown in Table 4 below. Specifically, the shrinkage is determined based on Formula (1) below:

Shrinkage=(Initial Length−Reduced Length)/Initial Length×100%

wherein,
Initial Length=length or diameter of the solid deodorizer before use
Reduced Length=length or diameter of the solid deodorizer after a predetermined time period of use.

TABLE 4

| | | | | Shrinkage | | |
|---|---|---|---|---|---|---|
| | | | | (Of Length/Diameter) | | % |
| Inventive | Sample | Initial Dimensions (first | Weight | at 45 Days after use | | Shrinkage |
| Sample | Shape | configuration before use) | (g) | (second configuration) | | at 45 days |
| 1 | Circle | Diameter = 40 mm | 5.8 | 4.2 | mm | 10.5 |
| | | Thickness = 5 mm | | | | |
| 2 | Oval | Length × Width × | 5.8 | 5.4 | mm | 10.0 |
| | | Thickness = | | | | |
| | | 54 × 26.5 × 5 mm | | | | |
| 3 | Oval | Length × Width × | 8.2 | 5.6 | mm | 10.4 |
| | | Thickness = | | | | |
| | | 54 × 26.5 × 5 mm | | | | |
| 4 | Rectangle | Length × Width = | 8.2 | 7 | mm | 12.5 |
| | | 56 × 32 × 5 mm | | | | |
| 5 | Rectangle | Length × Width = | 8.2 | 5 | mm | 10.2 |
| | | 49 × 27 × 5 mm | | | | |
| 6 | Rectangle | Length x Width = | 8.2 | 3.1 | mm | 6.3 |

49 × 27 × 5 mm

All the above Inventive Samples 1 to 6 have a shrinkage of 6.3% to 12.5%, i.e., less than 40%. As a result, each of the samples at the end of life is a single unitary piece having a size sufficient for easy removal and thereby reducing mess. In contrast, conventional gels have a gelling polymer which remains after water and perfume evaporates, and the remaining gelling polymer causes stickiness in the gel. Further, the remaining gel also stick to the container break easily and causes messy residue.

An example is described below:
A. A flexible solid deodorizer for releasably interlocking to a volatile composition dispenser, the solid deodorizer comprising: a first central evaporative surface, a second central evaporative surface, and a peripheral evaporative surface, wherein the peripheral evaporative surface circumscribes at least a part of the first and second central evaporative surfaces to define a side of the deodorizer; wherein the deodorizer is configured for releasably interlocking to the dispenser, wherein at least a portion of the deodorizer is configured to releasably interlock with at least a portion of the dispenser to form a solid surface substantially free of an opening.
B. The deodorizer according to paragraph A, wherein a shape of an outer profile of the solid deodorizer corresponds to an inner profile of the dispenser.
C. The deodorizer according to paragraph A or B, wherein the at least a portion of the deodorizer is configured for releasably locking to the at least a portion of the dispenser by one of: interference fit, pressure fit, friction fit, and combinations thereof.
D. The deodorizer according to any one of the preceding paragraphs, wherein a shape of the at least a portion of the deodorizer corresponds to a shape of the at least a portion of the dispenser, wherein the at least a portion of the deodorizer comprises an anchor receiving component, and the at least a portion of the dispenser comprises an anchor.
E. The deodorizer according to any one of the preceding paragraphs, wherein the solid deodorizer is capable of shrinkage relative to the peripheral evaporative surface from an initial size to a reduced size smaller than the initial size upon exposure to the environment in an interior space.
F. The deodorizer according to any one of the preceding paragraphs, wherein the solid deodorizer is non-aqueous, preferably comprises less than 1% by weight of water, more preferably substantially free of water.
G. The deodorizer according to any one of the preceding paragraphs, wherein the solid deodorizer comprises a gel composition selected from the group consisting of: an ethyl cellulose polymer, a chemically cross-linked polyol or derivative thereof, and mixtures thereof
H. The deodorizer according to any one of the preceding paragraphs, wherein the solid deodorizer comprises a chemically cross-linked polyol, wherein the polyol or derivative thereof is selected from the group consisting of: polyol, polyester polyol, polyglycerol and mixtures thereof, preferably the polyol derivative is a polyester polyol, more preferably castor oil, even more preferably the polyester polyol is cross-linked using a cross-linking agent selected from the group consisting of: isocyanates, isothiocynates and mixtures thereof.
I. A volatile composition dispenser comprising
(a) a solid deodorizer according to any one of the preceding paragraphs;
(b) a releasable interlock for releasably interlocking the solid deodorizer to the dispenser, wherein each of the first and second central evaporative surfaces are substantially free of contact with surfaces in the dispenser.
J. The dispenser according to paragraph I, wherein the dispenser comprises an inner profile having a shape corresponding to the outer profile of the solid deodorizer.
K. The dispenser according to paragraph I or J, wherein the releasable interlock is selected from the group consisting of: a releasable mechanical interlock, a releasable chemical interlock, and combinations thereof.
L. The dispenser according to paragraph K, wherein the dispenser comprises a releasable mechanical interlock, wherein the at least a portion of the dispenser comprises an anchor disposed within the dispenser; and the at least a portion of the deodorizer comprises an anchor receiving component located at the proximal end or distal end of the deodorizer.
M. The dispenser according to paragraph L, wherein a size and a shape of the anchor is configured to corresponding to a size and a shape of the anchor receiving component.
N. The dispenser according to any one of the preceding paragraphs L or M, wherein the dispenser comprises a housing comprising a housing inner surface, wherein the anchor is disposed on the housing inner surface.
O. The dispenser according to any one of the preceding paragraphs L or M, wherein the dispenser comprises an article holder, wherein the article holder comprises an opening characterized by a size corresponding to a size of the solid deodorizer, wherein the anchor is disposed on an inner surface of the opening.
P. A method of refilling a volatile composition dispenser with a solid deodorizer, the method comprising:
providing a volatile composition dispenser according to any one of paragraphs I to 0;
applying a pressure on one of the first central evaporative surface and the second central evaporative surface of the solid deodorizer to remove the solid deodorizer from the dispenser;
releasably interlocking a second solid deodorizer according to any one of paragraphs A to H to the dispenser.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. What is claimed is:

1. A flexible solid deodorizer for releasably interlocking to a volatile composition dispenser, the solid deodorizer comprising: a first central evaporative surface, a second central evaporative surface, and a peripheral evaporative surface, wherein the peripheral evaporative surface circumscribes at least a part of the first and second central evaporative surfaces to define a side of the deodorizer; wherein the deodorizer is configured for releasably interlocking to the dispenser, wherein at least a portion of the deodorizer is configured to releasably interlock with at least a portion of the dispenser to form a solid surface substantially free of an opening.

2. The deodorizer according to claim 1, wherein a shape of an outer profile of the solid deodorizer corresponds to an inner profile of the dispenser.

3. The deodorizer according to claim 1, wherein the at least a portion of the deodorizer is configured for releasably locking to the at least a portion of the dispenser by one of: interference fit, pressure fit, friction fit, and combinations thereof

4. The deodorizer according to claim 1, wherein a shape of the at least a portion of the deodorizer corresponds to a shape of the at least a portion of the dispenser, wherein the at least a portion of the deodorizer comprises an anchor receiving component, and the at least a portion of the dispenser comprises an anchor.

5. The deodorizer according to claim 1, wherein the solid deodorizer is capable of shrinkage relative to the peripheral evaporative surface from an initial size to a reduced size smaller than the initial size upon exposure to the environment in an interior space.

6. The deodorizer according to claim 1, wherein the solid deodorizer is non-aqueous, and comprises less than 1% by weight of water.

7. The deodorizer according to claim 1, wherein the solid deodorizer comprises a gel composition selected from the group consisting of: an ethyl cellulose polymer, a chemically cross-linked polyol or derivative thereof, and mixtures thereof.

8. The deodorizer according to claim 1, wherein the solid deodorizer comprises a chemically cross-linked polyol, wherein the polyol or derivative thereof is selected from the group consisting of: polyol, polyester polyol, polyglycerol and mixtures thereof.

9. A volatile composition dispenser comprising
a solid deodorizer according to claim 1;
a releasable interlock for releasably interlocking the solid deodorizer to the dispenser, wherein each of the first and second central evaporative surfaces are substantially free of contact with surfaces in the dispenser.

10. The dispenser according to claim 9, wherein the dispenser comprises an inner profile having a shape corresponding to the outer profile of the solid deodorizer.

11. The dispenser according to claim 9, wherein the releasable interlock is selected from the group consisting of: a releasable mechanical interlock, a releasable chemical interlock, and combinations thereof.

12. The dispenser according to claim 11, wherein the dispenser comprises a releasable mechanical interlock, wherein the at least a portion of the dispenser comprises an anchor disposed within the dispenser; and the at least a portion of the deodorizer comprises an anchor receiving component located at the proximal end or distal end of the deodorizer.

13. The dispenser according to claim 12, wherein a size and a shape of the anchor is configured to corresponding to a size and a shape of the anchor receiving component.

14. The dispenser according to claim 9, wherein the dispenser comprises a housing comprising a housing inner surface, wherein the anchor is disposed on the housing inner surface.

15. The dispenser according to claim 9, wherein the dispenser comprises an article holder, wherein the article holder comprises an opening characterized by a size corresponding to a size of the solid deodorizer, wherein the anchor is disposed on an inner surface of the opening.

16. The deodorizer according to claim 8, wherein the solid deodorizer comprises a polyol derivative comprising a polyester polyol.

17. A method of refilling a volatile composition dispenser with a solid deodorizer, the method comprising:
providing a volatile composition dispenser according to claim 9;
applying a pressure on one of the first central evaporative surface and the second central evaporative surface of the solid deodorizer to remove the solid deodorizer from the dispenser;
releasably interlocking a second solid deodorizer according to claim 1 to the dispenser.
